(19) United States
(12) Patent Application Publication
(10) Pub. No.: US 2010/0021944 A1
(43) Pub. Date: Jan. 28, 2010

(54) Method for Decreasing Interference in Results of Immunochemical Methods

(75) Inventors: Eric W. McIntush, Richards, TX (US); James M. Billings, JR., Willis, TX (US); Valarie J. Florack, The Woodlands, TX (US); Debra L. Garrett, Montgomery, TX (US); Henry F. Carwile, Montgomery, TX (US)

Correspondence Address: BURLESON COOKE L.L.P., 2040 NORTH LOOP 336 WEST, SUITE 123, CONROE, TX, 77304, US

(73) Assignee: Bethyl Laboratories, Inc., Montgomery, TX (US)

(21) Appl. No.: 12/547,658

(22) Filed: Aug. 26, 2009

Related U.S. Application Data
(63) Continuation of application No. 11/343,693, filed on Jan. 31, 2006, now abandoned.

(51) Int. Cl. G01N 33/53

(52) U.S. Cl. 435/7.9

(57) ABSTRACT

Methods and materials are provided for decreasing or eliminating interference from molecules resulting from upstream immunochemical procedures, such as Immunoprecipitation, that employ an Immunoglobulin Binding Molecule (an IBM, e.g., Protein A) in subsequent downstream methods, such as Western Blot. An effective amount of a ligand of the IBM is used to block the IBM or fragments of the IBM on a support membrane used in the downstream method. In another embodiment, a secondary antibody that is specific either for heavy chain or light chain antibody fragments or that recognizes intact antibody molecules but does not recognize individual antibody fragments may be employed with the ligand of the IBM.

This application is a continuation of application Ser. No. 11/343,693 filed Jan. 31, 2006.

### BACKGROUND OF THE INVENTION

1. Field of the Invention

This invention relates to immunochemical analytical methods. More particularly, in methods including a first and a second immunochemical technique to characterize an antigen, methods are provided for decreasing interference from molecules derived from the first technique, such as immunoglobulin binding molecules and fragments of antibodies, in the second technique.

2. Description of Related Art

As the era of genomics concludes a looming challenge to biology and medicine is to understand how the influences of individual components are integrated to dictate the ultimate behavior of biological systems. Proteins are a primary class of components of biological systems, and understanding the interrelated influences of individual proteins on biological systems is the primary goal of a field of science commonly referred to as Proteomics. Pre-proteomic approaches to the study of proteins have focused on determining the quantity and location of individual proteins. In these studies, the existence of multi-protein complexes was seldom investigated.

In contrast, proteomic approaches now accept the premise that proteins in biological systems do not exist individually, but that each individual protein exists as a member of a heterogeneous population of molecules. In order to understand the integration of all the influences that dictate the ultimate behavior of biological systems, the interactions among the members of the heterogeneous population must be understood. A goal of proteomic studies is to define the interactions and their integration in normal and pathological situations.

Most intracellular proteins participate in multi-protein complexes. It appears likely that individual molecules of a given protein participate in different, functionally distinct multi-protein complexes. The specific function of each protein complex is determined by the participating members of that complex. In addition, the multi-protein complexes in which a given target protein participates is likely to change as the requirements of the system change. Within a cell, a protein may participate in one set of multi-protein complexes as the system responds to one stimulus, and the same protein may participate in a distinctly different set of multi-protein complexes as the system responds to a different stimulus.

A common initial step in investigating the function of a protein of interest is to evaluate the multi-protein complexes in which it participates. In order to evaluate the multi-protein complexes, they must be isolated. Therefore, a source of the protein complexes and a means of isolating those that contain the protein of interest are required. Common crude preparations containing multi-protein complexes are cell and tissue lysates, which are solutions containing the soluble components isolated from cells or tissue, respectively. The target protein and the proteins with which it associates are often isolated using upstream immunochemical procedures. Immunochemical procedures are those that utilize antibodies (immunoglobulin molecules selected based upon an ability to bind specifically to the protein of interest). Proteins of interest are isolated by upstream immunochemical procedures that can be characterized as those that purify the protein of interest using an antibody that has specific affinity for the protein of interest. On a small-scale such affinity purification of the protein of interest using a specific antibody is commonly referred to as immunoprecipitation (herein “IP”) (Kessler S. W., 1975, J Immunol 115: 1617-1624; Harlow and Lane, Antibodies: A Laboratory Manual, Cold Spring Harbor Laboratory Press, Cold Spring Harbor, N.Y., 1988; Harlow and Lane, Using Antibodies: A Laboratory Manual, Cold Spring Harbor Laboratory Press, Cold Spring Harbor, N.Y., 1999). On larger scales such affinity purification of the protein of interest using a specific antibody is commonly known as immunoaffinity purification (“IAP”) (Antibodies: A Laboratory Manual, 1988; and Harlow and Lane, Using Antibodies: A Laboratory Manual, 1999).

Subsequent to purification of the multi-protein complex using the upstream immunochemical procedure, the components are analyzed by downstream immunochemical procedures. The most common downstream immunochemical procedure is Western Blotting (WB, a.k.a. immunoblotting) (Renart J., Reiser J., Stark G. R., 1979, Proc Natl Acad Sci U.S.A. 76:3116-20; Towbin H., Staehelin T., Gordon J., 1979, Proc Natl Acad Sci U.S.A. 76:4350-4; Burnette W. N., 1981, Anal Biochem. 112:195-203). If the limitations that require separation of the components based upon their chemical and/or physical properties (as is done for WB) were eliminated, the plausible downstream immunochemical procedures would expand to include such procedures as Dot Immunoblotting (i J., Sentenac A., Fromageot P., 1982, J. Biol Chem. 257:2613-8; Harlow and Lane, Antibodies: A Laboratory Manual, 1988) and other immunochemical procedures in which the sample derived from the upstream immunochemical procedure is bound to a solid support.

In order to understand the limitations to downstream immunochemical procedures, one must first consider the upstream immunochemical procedures (e.g. IAP and IP), the molecules that they introduce to the samples and the impact of these introduced molecules on the evaluation of the samples by downstream immunochemical procedures.

In addition to antibodies, the upstream immunochemical procedures utilize an Immunoglobulin Binding Molecule (hereafter “IBM”) such as Protein A or Protein G (Langone J. J., 1982, Adv Immunol. 32:157-252; Harlow and Lane, Antibodies: A Laboratory Manual, 1988)]. The IBM is used to bind the antibody, and a solid support is used to bind the IBM. Thus, a complex is formed. The complex that is the result of the upstream immunochemical procedure can be conveniently depicted as Associated Proteins-Protein of Interest-Antibody-IBM-Solid Support. To form the complex, the cell or tissue lysate is exposed to antibodies under conditions in which the antibodies bind specifically to the protein of interest. The antibody-protein complex is isolated by binding of the antibody to an IBM. The antibody may be captured by the IBM before or after exposing the lysate to the antibody. Likewise, the IBM may be bound to the solid support before or after exposing the antibody to the IBM.

Once formed, the complex of Associated Proteins-Protein of Interest-Antibody-IBM-Solid Support is washed in order to remove nonparticipating components of the lysate. Thereafter, the components are dissociated to release the protein of interest and the proteins with which it associates. The dissociated components are the sample to be analyzed by downstream immunochemical procedures.

Owing to the strong interaction between the protein and antibody, efficient dissociation of these two components of the complex requires exposure to harsh conditions in order to efficiently dissociate the protein of interest from the antibody. A result of using harsh conditions is that other components (namely antibody molecules, fragments of antibody molecules, IBM and fragments of IBM) of the complex are also dissociated. Thus, exposure to harsh conditions: 1) releases the associated proteins from the protein of interest, 2) releases the protein of interest from the antibody molecules, 3) fragments the antibody molecule into its component parts (heavy and light chain antibody fragments), 4) releases the antibody fragments from the IBM, and 5) releases some IBM and fragments thereof from the solid support. Ultimately, all of these products are present in the sample to be analyzed by downstream immunochemical procedures, but only two of them (associated proteins and protein of interest) are desirable. The other products (antibody molecules, fragments of antibody molecules, IBM and fragments thereof) are undesirable owing to the “noise” that they contribute using established downstream immunochemical procedures. (“Noise” is a word used herein to describe the interference in detection of a target molecule or protein of interest by the presence of other molecules that cause a response in the detection method that is not clearly distinguishable from the response of the target molecule.)

Despite their limitations, downstream immunochemical procedures have two advantages: 1) high throughput—efficient analysis of large numbers of samples, and 2) quantitative results—the amount of signal from different samples is proportional to the amount of the protein of interest. On balance, these advantages outweigh the limitations imposed on downstream immunochemical procedures by the noise contributed by the contaminating antibody fragments, IBM and fragments thereof. Improvements that reduce noise in existing downstream immunochemical procedures will increase the breadth and efficacy of downstream immunochemical procedures.

Western Blotting (WB) is the most common downstream immunochemical procedure used to analyze components of multi-protein complexes. In WB, the components of the samples (products of the upstream immunochemical procedure in this instance) are separated by electrophoresis in a polyacrylamide gel, electrophoretically transferred to a solid support, the solid support bound components are exposed to an antibody that binds to a specific protein, and the amount of antibody that is bound is determined. Most commonly, the amount of antibody (termed primary antibody) bound to the specific protein is detected using an antibody (termed secondary antibody) that binds specifically to immunoglobulins of the species in which the primary antibody was raised. The use of a secondary antibody provides two benefits: 1) amplification of signal—secondary antibodies bind at multiple locations on the primary antibody, and 2) broad utility—a single preparation of secondary antibodies can be used in combination with primary antibodies to any protein of interest so long as the primary antibody is an immunoglobulin from the species that is recognized by the secondary antibody. Therefore, manipulations such as bonding to a reporter molecule (a molecule such as an enzyme or a fluorochrome used for detection) can be undertaken with a single secondary antibody, and the resulting reagent can be used in combination with any primary antibody raised in a given species. This eliminates the need to perform the manipulations on each primary antibody. Use of a secondary antibody also allows the possibility of utilizing a secondary antibody that is sensitive only to light chain antibody fragments, heavy chain antibody fragments, intact or native antibodies or both heavy and light chains.

An advantage of the use of WB as a downstream immunochemical procedure is that the components of the sample (products of the upstream immunochemical procedure) are separated in a polyacrylamide gel. Migration of the components through the polyacrylamide gel is dictated by the physical and chemical properties of the individual components. Thus many of the components of the sample to be analyzed possess physical and chemical properties that allow them to be physically separated from the components (antibody fragments, IBM and fragments thereof) of the sample that contribute to noise.

Provided that the signal for the protein of interest is strong and the protein of interest migrates distally to the migration of the contaminants (antibody fragments, IBM, and fragments thereof) in polyacrylamide, the signal will likely be distinguishable from the noise, and useful data are likely to be obtained. If the signal for the protein of interest is not strong or the protein of interest migrates proximally to the migration of the contaminants in polyacrylamide, the noise associated with the contaminants is likely to mask the signal, and useful data are not likely to be obtained. An example of a signal that is obscured by noise is illustrated in Panel A of FIG. 1. The sample for Lane 3 of Panel A of FIG. 1 is the product from a negative control IP reaction that used normal rabbit immunoglobulin rather than a specific antibody. In this lane, the background noise (caused by heavy- and light-chain fragments of antibody molecules, protein A and fragments of protein A) inherent in the conventional procedures of applying downstream immunochemical procedures to analyze the product of an upstream immunochemical procedure can be seen. The samples for lanes 1 and 2 are the products of IP reactions using a specific antibody. While some signal is apparent in lane 1, it cannot be readily distinguished from noise, and the data are not useful. There is virtually no signal in lane 2 that can be distinguished from the noise.

Presently available techniques and products that attempt to minimize the impact of noise have not been widely accepted owing to substantial deviations from the conventional reagents and methods that are used for upstream and/or downstream immunochemical procedures. SEIZE X® from Pierce Chemical of Rockford, Ill. attempts to minimize the amount of antibody fragments, IBM and fragments thereof that are released in the final step of the upstream immunochemical procedure. This product uses a cross-linker, disuccinimidyl suberate, to cross-link primary antibody to the IBM (Protein-A or Protein-G) on an agarose bead. An additional modification to common practices is that the protein of interest is released from the Protein of Interest-Antibody-IBM-Solid Support complex using acidic conditions that do not disrupt the cross-linked Antibody-IBM-Solid Support complex. This technique allows the possibility of reusing the primary antibody and reducing heavy and light chain contamination in the final sample. The SEIZE X kit's disadvantages include factors such as: 1) increased time, 2) increased complexity, and 3) reduced efficiency.

TrueBlot™ from eBiosciences of San Diego, Calif. attempts to minimize the amount of noise contributed by IBM and fragments thereof that are released in the final step of the upstream immunochemical procedure by replacing conventional IBM (Protein-A, Protein-G or Protein-A/G) with an alternative IBM (an antibody). (See also: Pat. Pub. No. US 2005/0142609) In addition, this product attempts to minimize the amount of noise contributed by antibody fragments that are released in the final step of the upstream immunochemical procedure by replacing conventional secondary detection antibodies with secondary detection antibodies that recognize intact antibody molecules but do not recognize individual antibody fragments. This provides an advantage over previous methods, but the use of highly efficient IBM (e.g. Protein-A, Protein-G or Protein A/G) is incompatible with this method owing to continued contribution of noise by these IBMs if they were to be used.

Materials and methods for reducing noise are needed in order to distinguish a broader range of signals. In addition, there is a need to reduce the noise to such degree that separation of the components of the sample by polyacrylamide gel electrophoresis would not be necessary. More sensitive, higher throughput, less costly and less labor intensive downstream immunochemical procedures may then be utilized.

### SUMMARY OF INVENTION

Materials and methods are provided for improving immunochemical techniques for proteins. Effects on a downstream technique from interfering molecules resulting from an upstream technique employing an Immunoglobulin Binding Molecule (IBM) are decreased or eliminated by addition of a ligand of the IBM to the solid support used in the downstream technique. In the method of Immunoprecipitation followed by Western Blot, the procedure disclosed herein includes the step of adding a ligand of the IBM (Protein A, e.g.) to block the noise that is created by the IBM or fragments thereof that interferes with detection of a protein of interest. In another embodiment, a secondary antibody having a reporter molecule is used that is responsive only to heavy-chain or light-chain fragments, both heavy- and light-chain fragments of antibodies or that is not responsive to antibody fragments.

### BRIEF DESCRIPTION OF THE DRAWINGS

FIG. 1 shows results of Western Blotting with:

Panel A—standard procedures for blotting the sample of an immunoprecipitation reaction;
Panel B—an alternative secondary antibody, which recognizes the light chain antibody fragments but not the heavy chain antibody fragments;
Panel C—use of a ligand of an IBM (hereafter referred to as an LIBM) to saturate the IBM and thereby block its activity by mass action; and
Panel D—the combined use of an alternative secondary antibody, which recognizes the light chain antibody fragments but not the heavy chain antibody fragments, and an LIBM to saturate the IBM and thereby block its activity by mass action.

### DETAILED DESCRIPTION

Terms used herein have the following meanings:

An Immunoglobulin Binding Molecule (IBM) is a molecule that binds to immunoglobulins. Examples are Protein-A and Protein-G.

A Ligand of an Immunoglobulin Binding Molecule (LIBM) is any molecule that occupies the immunoglobulin binding site of an IBM or fragments thereof. An example is pig IgG or sheep IgG.

An upstream immunochemical procedure is one that: 1) uses immunoglobulins, 2) uses an IBM, and 3) produces a sample that is used in a downstream immunochemical procedure. An example is immunoprecipitation.

A downstream immunochemical procedure is a technique that uses immunoglobulins and is performed using a sample from an upstream immunochemical procedure. An example is Western Blotting.

A primary antibody is an immunoglobulin raised and selected based upon specific binding to a protein of interest. An example is an antibody raised in rabbits that recognizes the human protein known as histone deacetylase 3.

A secondary antibody is an immunoglobulin raised in a species that specifically binds immunoglobulins and fragments thereof of another selected species. An example is an antibody raised in goats that recognizes rabbit immunoglobulins.

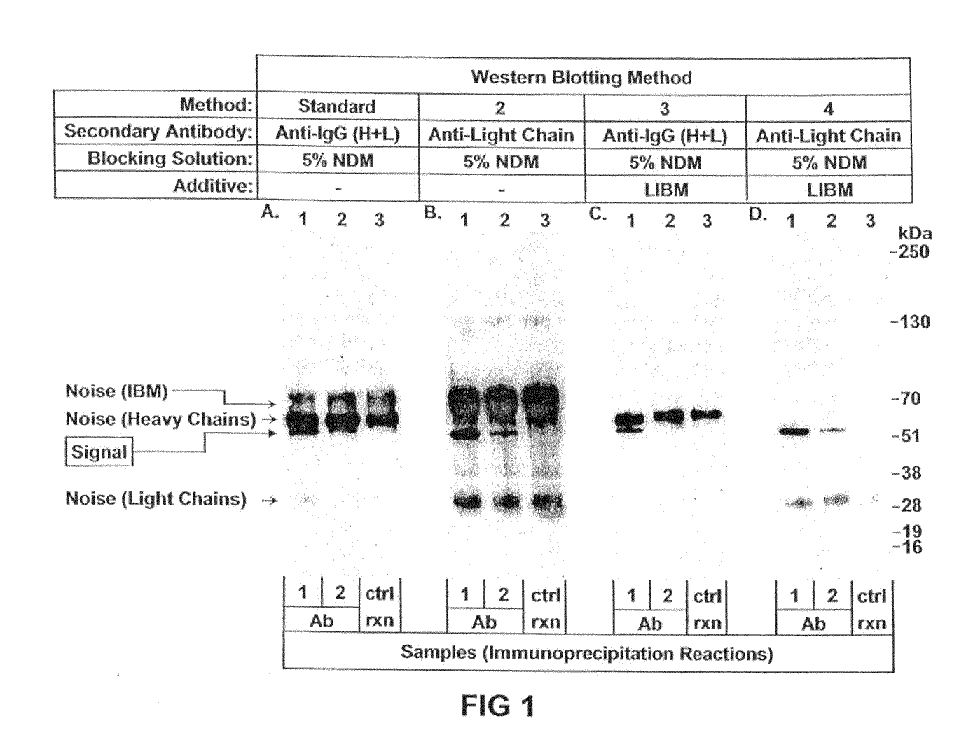

Referring to FIG. 1, results are shown in Panel A from using the standard Western Blotting Method (WB) to analyze a sample containing a known target protein with two different antibodies (Lanes 1 and 2) and a control sample (Lane 3) The procedures used to produce the results in Panel A were as follows:

1. Preparation of Cell Lysate used for Immunoprecipitation:
a. Cells (HeLa cells) were grown to approximately 80% confluence (approximately 20% of the available surface not covered by cells) on 100 mm polystyrene tissue culture plates.
b. The adherent cells were washed two times in ice cold PBS (phosphate buffered saline which consisted of 50 mM phosphate pH 7.2 and 150 mM NaCl).
c. Cells were lysed to release soluble cellular proteins using 0.5 ml of lysis buffer [250 mM NaCl, 50 mM Tris, 5 mM EDTA, 0.05% (vol:vol) Tween-20) containing inhibitors of proteases per 100 mm plate. The cells were scraped from the plates, transferred to reaction vessels (1.5 ml micro-centrifuge tubes), and the reaction vessels were placed on ice for 30 minutes to assure efficient lysis.
d. The reaction vessels were centrifuged (10,000×g; 5 minutes), and the supernatant was used for the immunoprecipitation reaction.

2. Procedures for Immunoprecipitation Reaction:
a. The immunoprecipitation reactions were:

| | Ab1 | Ab2 | ctrl rxn |
|---|---|---|---|
| Sample: | 0.5 ml | 0.5 ml | 0.5 ml |
| | cell lysate | cell lysate | cell lysate |
| Primary Antibody: | 3 mcg | 0.6 mcg | — |
| Normal Rabbit IgG: | — | 2.4 mcg | 3 mcg |
| Lane in Panels: | 1 | 2 | 3 |

In brief, 0.5 ml aliquots of cell lysate were placed in reaction vessels to which were added 3 mcg (Ab1 reaction; lane 1 of each panel of FIG. 1) or 0.6 mcg plus 2.4 mcg rabbit immunoglobulin (Ab2 reaction; in lane 2 of each panel of FIG. 1) of a rabbit antibody to human Histone Deacetylase 3 (a.k.a. HDAC3) as the immunoprecipitating antibody. Normal rabbit IgG (ctrl r×n; lane 3 in each panel of FIG. 1) was used in a negative control reaction to obtain a measure of the background noise inherent to the procedure of immunoprecipitation. Subsequently, 100 mcl aliquots of a 20% slurry of Protein-A agarose beads (Amersham Biosciences, Piscataway, N.J.) were added to the reaction vessels. The reactions were allowed to proceed for 3 hours at 4° C., during which time the reaction vessels were continually rotated end-over-end to assure thorough mixing.
b. At the end of the reaction, the products of the immunoprecipitation reactions were washed. To accomplish this, the reaction vessels were centrifuged (500×g; 5 minutes), the supernatants were aspirated, and the precipitants representing the product of the reaction were washed by suspension in ice cold lysis buffer (without protease inhibitors which would contaminate the final reaction product). The steps of centrifugation/aspiration/suspension were repeated twice.
c. Following the last wash of the reaction products, the reaction vessels were centrifuged (500×g; 5 minutes), the supernatants were aspirated and discarded, and the components were dissociated with 40 mcl of SDS-PAGE loading buffer (PAGEgels, San Diego, Calif.).

3. Separation of Reaction Products in a Polyacrylamide Gel:
Eight mcl of the reaction product from the immunoprecipitation reaction were separated by electrophoresis in a 4-12% Tricine Gel (PAGEgels, San Diego, Calif.).

4. Electrophoretic transfer from Polyacrylamide Gel to Solid Support:
Proteins within the gel were electrophoretically transferred to a solid support (nitrocellulose membrane; Invitrogen, Carlsbad, Calif.).

5. Procedures for Western Blotting:
a. The membrane was placed in Diluent A [5% (wt:vol) non-fat dry milk/0.05% (vol:vol) Tween-20 in PBS] for one hour at room temperature to saturate the un-reacted protein binding sites on the solid support with protein from the non-fat dry milk.
b. The membrane was placed in a primary antibody solution, which consisted of a rabbit antibody to human Histone Deacetylase 3 prepared by dilution in Diluent A to the optimal concentration (1 mcg/ml). The membrane was incubated in the primary antibody solution overnight at room temperature.
c. The membrane was washed (three times for 10 minutes each) in PBS containing 0.05% Tween-20.
d. Following the final wash of the membrane, it was transferred to a solution of secondary antibody diluted in Diluent A.

Secondary antibodies are available from a variety of commercial sources. The secondary antibody used was chosen owing to its reactivity with the immunoglobulins of the species in which the primary antibody was raised. The primary antibody was raised in rabbits. Therefore, the secondary antibody was one that bound to rabbit immunoglobulins. More specifically, the secondary antibody used was goat anti-rabbit IgG (H+L chain) bonded to the reporter molecule horseradish peroxidase. The bonding to horseradish peroxidase was selected owing to compatibility with the final detection step, which was chemiluminescent detection using a reagent that is enzymatically converted by horseradish peroxidase.
e. The membrane was incubated (one hr; room temperature). Thereafter, the membrane was washed (three times for 10 minutes each) in PBS containing 0.05% Tween-20.
f. The membrane was developed using SuperSignal West Dura Substrate (Pierce Biotechnology, Rockford, Ill.). The signal was visualized using a CCD camera (UVP,Inc. Upland, Calif.).

Panel A illustrates the difficulty of recognizing the signal, which characterizes the molecular weight of the protein of interest. In this example, the protein of interest (from the lysate of the HeLa cells) exhibited a molecular weight of about 51 kDa, which was near the masking noise, which was believed to result from antibody fragments and the IBM (Protein-A) or fragments of the IBM.

Panels B, C and D of FIG. 1 resulted from modifications to the procedures used for the Standard Method (Panel A). The modified procedures used were as follows:
1. Procedures for preparation of cell lysates used for immunoprecipitation, the immunoprecipitation reaction, separation of reaction products in a polyacrylamide gel and electrophoretic transfer from the gel to the solid support for Methods 2, 3 and 4 were the same as for the Standard Method
2. For Method 2 a single change from the Standard Method was made to the procedures for Western Blotting. The change consisted of replacing the goat anti-rabbit IgG (H+L chain) bonded to horseradish peroxidase with an antibody that is specific for Light Chains of rabbit immunoglobulins (goat anti-rabbit Light Chain antibody) bonded to horseradish peroxidase.
3. For Method 3 a single change from the Standard Method was made to the procedures for Western Blotting. The change consisted of adding a LIBM to Diluent A. The LIBM used was normal sheep IgG used at a concentration of 1 mg/ml.
4. For Method 4, the changes used for Methods 2 and 3 were combined. The two changes were: 1) the goat anti-rabbit IgG (H+L chain) bonded to horseradish peroxidase was replaced with an antibody that is specific for light chains of rabbit immunoglobulins (goat anti-rabbit light chain antibody) bonded to horseradish peroxidase, and 2) a LIBM was added to Diluent A. The LIBM used was normal sheep IgG.

The results shown in Panel B of FIG. 1 illustrate that the noise contributed by contaminating antibody fragments can be significantly reduced by using a secondary antibody that recognizes the light chain antibody fragments (around 30 Kda) but not the heavy chain antibody fragments. The secondary antibody that binds to rabbit immunoglobulin light chains was prepared by injecting goats with rabbit immunoglobulin mu, which contains the light chain molecules common to all rabbit immunoglobulins. Thereafter, the antibodies to light chains were affinity purified by adsorption to the light chains of rabbit immunoglobulin gamma. The secondary antibody was bonded to horseradish peroxidase using established procedures (Hermanson, G. T., Bioconjugate Techniques Academic Press, San Diego, 1996). The ratio of signal to noise at the location of the protein of interest (about 51 kDa) was much better when using the alternative secondary antibody (Panel B) than when using the standard method for WB (Panel A). The signals, which are present in lanes 1 and 2 of Panel B, are more readily distinguished from the noise, which is present in all lanes of Panels A and B of FIG. 1.

To eliminate the noise associated with contaminating Protein-A and fragments thereof, the Protein-A was exposed to saturating concentrations of a sheep IgG as LIBM to block the ability of Protein A to bind the primary or secondary antibody used in the WB procedure. This strategy was effective and is illustrated by comparing Panel A (not blocked) to Panel C (blocked with the LIBM). The concentration of Sheep IgG used was 6.6×10⁻⁶ molar. The effective range of concentrations of the LIBM is very broad—from about 1×10⁻⁸ molar to greater than 1×10⁻³ molar. The effective range is, in part, a function of 1—the concentration at which the primary and secondary antibodies are used, and 2—the affinity of the IBM for the LIBM relative to the affinity of the IBM for the primary and secondary antibodies used in the downstream immunochemical procedure. The LIBM should be used at concentrations that block binding of the primary and secondary antibodies to the IBM through binding of the LIBM to the IBM.

To eliminate both the noise associated with contaminating antibody heavy chain fragments and IBM and fragments thereof, the Protein-A was exposed to saturating concentrations of sheep IgG to block its ability to bind the antibody in the downstream immunochemical procedure and an alternative secondary antibody, which recognizes the light chain antibody fragments but not the heavy chain antibody fragment, was used. The strategy was effective as illustrated in Panel D of FIG. 1. The concentration of Sheep IgG used was 6.6×10⁻⁶ molar. The effective range of concentrations of the LIBM is discussed above.

An IBM may be a naturally occurring molecule that binds to immunoglobulins or a synthetic molecule that binds to immunogloublins. Commonly used IBMs are Protein-A and Protein-G. Alternative IBM may be identified by a method that determines binding to immunoglobulins. It may be identified by analysis of materials selected from a library of synthetic compounds, an engineered protein, a compound made in a mammalian system, a polymer made in a non-mammalian system, a compound prepared by recombinant DNA technology, a polymer made by E. coli phage display, a protein scaffold library, a peptide display library, a directed evolution library, or a protein array-based library.

An LIBM is any molecule that occupies the immunoglobulin binding site of an IBM or fragments thereof. Other LIBMs that were tested and proven to be effective using the procedures described above include effective concentrations of serum from pigs, partially purified IgG from pigs and purified IgG from pigs. Each one eliminated the noise contributed by contaminating Protein-A or fragments of Protein-A. The secondary antibody must not bind the LIBM. Those used to date (purified sheep IgG and pig IgG) are not bound by the goat anti-rabbit light chain secondary antibody described and used above.

An LIBM may be a naturally occurring molecule or a synthetic molecule. An LIBM includes those molecules selected from a group consisting of an immunoglobulin, a portion of an immunoglobulin, a fragment of an immunoglobulin, a variant of an immunoglobulin, an engineered protein, a polymer scaffold, an engineered compound, a polypeptide, a compound made in a mammalian system, a polymer made in a non-mammalian system, and a polymer made in an E. coli phage display.

A naturally occurring LIBM may be isolated from animals including mammals selected from the group of humans, non-human primates, rabbits, mice, rats, rodents, guinea pigs, swine, sheep, goats, horses, donkeys, cattle, whales and dolphins. A preferred LIBM is serum, purified or partially purified IgG from pig. An LIBM may be isolated from non-mammmals selected from the group of birds, reptiles and fish.

Novel LIBMs may be identified by using an IBM in methods that identify molecules that compete for the immunoglobulin binding site on the IBM. Such novel LIBMs are identified by analysis of materials selected from the group of a library of synthetic compounds, an engineered protein, a compound made in a mammalian system, a polymer made in a non-mammalian system, a compound prepared by recombinant DNA technology, a polymer made by E. coli phage display, a protein scaffold library, a peptide display library, a directed evolution library, and a protein array based library.

An upstream immunochemical procedure uses immunoglobulins, uses an IBM, and produces a sample that is used in a downstream immunochemical procedure. An upstream immunochemical procedure may be immunoprecipitation or immunoaffinity purification.

The example of a downstream immunochemical procedure discussed above is Western Blot. An alternative downstream immunochemical procedure uses the chemical and/or physical properties of a sample from an upstream immunochemical procedure to bind the components of the sample on a solid support. A solid support may be a bead, a plate, a sheet, a strip, a well or a tube. An example is the binding of the sample from an upstream immunochemical procedure to a derivitized polymer designed to immobilize the components of the sample. The immobilized sample is then exposed to an antibody, and the amount of antibody that bound the sample is determined. An example of an alternative downstream immunochemical procedure is dot immunoblotting, in which a sample from an upstream immunochemical procedure is applied directly onto nitrocellulose or other suitable, protein-binding support, the bound components are exposed to a primary antibody and the amount of primary antibody that binds is determined. Examples of other suitable, protein binding supports are polyvinylidene difluoride and nylon membranes.

A primary antibody is an immunoglobulin that binds specifically to a protein or proteins of interest. The primary antibody may be directly or indirectly linked to a reporter molecule. Linkage may be through covalent or non-covalent bonds. Reporter molecules may be enzymes, horseradish peroxidase, alkaline phosphatase, fluorescent proteins, biotin, avidin, streptavidin, radioactive isotopes, fluorochromes, fluorophores, dyes, and stains.

A secondary antibody is an antibody that recognizes the immunoglobulins of the species in which the primary antibody was generated. In most instances the secondary antibody is raised in a species other than that used to raise the primary antibody. An example of a secondary antibody is an antibody isolated from the serum of goats that binds specifically to rabbit immunoglobulins. A secondary antibody may be isolated from the serum of goats that will bind specifically to the light chain of rabbit immunoglobulins, for example. A secondary antibody may also be isolated from the serum of goats that will bind specifically to the heavy chain of rabbit immunoglobulins, for example. Also, it is known that a secondary antibody may be produced that binds specifically to native or intact immunoglobulins or both heavy and light-chain portions of immunoglobulins molecules. A secondary antibody may be directly or indirectly linked to a reporter molecule through covalent or non-covalent bonds. Reporter molecules may be enzymes, horseradish peroxidase, alkaline phosphatase, fluorescent proteins, biotin, avidin, streptavidin, radioactive isotopes, fluorochromes, fluorophores, dyes, and stains.

As an alternative to using a secondary antibody an IBM linked a reporter molecule may be used. The IBM linked to a reporter molecule may be Protein-A or Protein-G, for example. The IBM may be directly or indirectly linked to a reporter molecule through covalent or non-covalent bonds. Reporter molecules may be enzymes, horseradish peroxidase, alkaline phosphatase, fluorescent proteins, biotin, avidin, streptavidin, radioactive isotopes, fluorochromes, fluorophores, dyes or stains.

A kit may be prepared with packaging material and the materials disclosed herein for performing immunochemical analyses. The kit may include only an LIBM selected for use with a specific IBM. The kit may also include an LIBM and one or more additional components. The additional components may include an IBM, a solid support on which to immobilize a sample from an upstream immunochemical procedure, dry solids, solutions, primary antibodies, secondary antibodies, reporter molecules and combinations thereof. For example, the kit may contain: 1—a mixture of non-fat dry milk of bovine origin and serum proteins from pigs and 2—a secondary antibody that is specific for the light chain fragments of rabbit immunoglobulins. In another example a kit may contain 1—a preparation of purified immunoglobulin of rabbit origin, 2—a primary antibody raised in goats, and 2—a secondary antibody that is specific for the heavy chain fragments of goat immunoglobulin gamma. In another example a kit may contain 1—An IBM that may be Protein-G immobilized on a solid support, 2—an antibody for use in preparation of a sample by use of an immunoprecipitation reaction, 3—a piece of nitrocellulose on which to immobilize a sample, 4—a dry solid that is a preparation of purified immunoglobulin of sheep origin mixed with non-fat dry milk of bovine origin, 5—a solution for dissolving dry solids, 6—a primary antibody raised in mice, and 7—a secondary antibody that is specific for the light chain fragments of mouse immunoglobulins. A kit may also include instructions for use of the materials.

While particular preferred embodiments of the present invention have been described, it is not intended that these details should be regarded as limitations upon the present invention, except as and to the extent that they are included in the following claims.

What we claim is:

1. An immunochemical analytical method that decreases interference from molecules derived from a first step comprising:
isolating a protein of interest using immunoprecipitation or immunoaffinity purification procedures including the use of an IBM to bind the antibody; and
subjecting the sample obtained as a result of the immunochemical procedure to a Western Blotting procedure, wherein a ligand of the IBM is added to the solid support utilized in the Western Blotting technique.

2. The method of claim 1 wherein the IBM comprises Protein A or Protein G.

3. The method of claim 1 wherein the ligand comprises a molecule that occupies the immunoglobulin binding site of an IBM or fragments thereof.

4. The method of claim 1 wherein the ligand is isolated from animals.

5. The method of claim 1 wherein the ligand comprises pig IgG.

6. The method of claim 1 wherein the ligand is selected from the group of compounds consisting of an immunoglobulin, a portion of an immunoglobulin, a fragment of an immunoglobulin, a variant of an immunoglobulin, an engineered protein, a polymer scaffold, an engineered compound, a polypeptide, a compound made in a mammalian system, a polymer made in a non-mammalian system.
